(19) United States
(12) Patent Application Publication
(10) Pub. No.: US 2024/0091117 A1
(43) Pub. Date: Mar. 21, 2024

(54) SKIN-CLEANSING PRODUCT AND METHOD FOR OBTAINING SAID PRODUCT

(71) Applicant: ECZACIBASI TUKETIM URUNLERI SANAYI VE TICARET ANONIM SIRKETI, Beykoz, Istanbul (TR)

(72) Inventors: Urun KANDEMIRER, Çayirova, Kocaeli (TR); Gulcin Almina FIDANLIK, Çayirova, Kocaeli (TR)

(21) Appl. No.: 18/254,904

(22) PCT Filed: Nov. 5, 2021
(86) PCT No.: PCT/TR2021/051142

§ 371 Date: May 30, 2023

(30) Foreign Application Priority Data
Nov. 30, 2020 (TR) 2020/19264

(51) Int. Cl. A61K 8/365; A61K 8/02; A61K 8/67; A61Q 19/00; A61Q 19/10

(52) U.S. Cl. CPC A61K 8/365; A61K 8/0208; A61K 8/676; A61Q 19/005; A61Q 19/10

(57) ABSTRACT

Disclosed is a skin-cleansing product, which contains no antimicrobial agent and is suitable for use even in the premature infants. A method for obtaining the product is also disclosed. The skin-cleansing product is suitable for use even in the neonates and/or premature infants wherein the skin-cleansing product does not contain any antimicrobial agent for killing and/or preventing the growth of microorganisms.

### SUBJECT OF THE INVENTION

The invention relates to a skin-cleansing product, which contains no antimicrobial agent and is suitable for use even in the premature infants, and to a method for obtaining said product.

### STATE OF THE ART

The skin has the characteristics of a cover system for the human body, being in contact with the external environment and protecting the internal and external organs from the adverse environmental conditions. The degree of sensitivity of the skin is a property that varies from one person to another. The skin of the infants consists of a layer that is thinner than that of the adults and is therefore more sensitive.

The average duration of pregnancy is accepted as 280 days, i.e. 40 weeks. The births taking place in or before the 37^{th} week are considered preterm delivery and the infants delivered preterm are classified as premature. Every year, about 13 million infants are born as premature in the world. Since the premature infants have not yet achieved sufficient development, they need special care. Since the brown fatty tissues have not developed in the premature infants, they do not have the ability to preserve the body temperature, they may experience breathing and nutrition problems, and they carry the risk of infection. The care for the premature infants is provided at the intensive care units of the hospitals, in sterile incubators under the conditions of fixed temperature and humidity stimulating those of the uterus. In this period, the cleansing of the skin of the premature infant is generally performed with sterile cotton and pure water.

It is necessary to continue the care in a meticulous and sterile manner for the premature infants and the term infants after they are discharged from the hospital. Especially in cases where the umbilical cord has not yet fallen off, that the umbilical cord stays moist will delay its fall and this will in turn increase the infection risk. For this reason, a time should be allowed to pass until the umbilical cord dries and falls off, before giving an infant his/her first bath.

The pure water obtained by boiling and cooling the water and a clean cotton or cotton cloth/towel are used also in general for cleansing the skin of the infant in the period before giving the infant his/her first bath and/or in the period between two bathes. The necessity to understand and totally eliminate the textile chemicals contained in the clothes or towels used as well as to wash them at an elevated temperature following every use renders the process quite difficult and inconvenient. In addition to the process being difficult and inconvenient, it is not possible to make sure if this sterilization process performed under the household conditions is adequate. The inability to sufficiently sterilize the water and/or the cotton or cloth used not being sufficiently clean increases the risk of infection for the infants.

As an alternative, the disposable wet wipes are available, which are preferred for this purpose. However, said wet wipes contain various preservative chemicals in order to ensure their shelf life and preserve the effectiveness of their ingredients for a long time. Further, many available wet wipes include different chemicals like a surfactant and a perfume as ingredients, in addition to the preservative. Of such products, even those with the lowest content of chemicals contain at least one antimicrobial preservative. It is very likely for such substances to lead to adverse effects on the skin of the premature infants or neonates and the use of such type of wet wipes is not preferred for the premature infants and especially for the infants whose umbilical cord has not yet fallen off.

The need has arisen to perform an innovation in the relevant art in order to solve the above-mentioned problems. With this invention, a skin-cleansing product, which contains no antimicrobial preservative and is suitable for use even in the premature infants, and a method for obtaining said product, are provided.

### OBJECT OF THE INVENTION

An object of the invention is to develop a sterile skin-cleansing product, which contains no antimicrobial agent and is suitable for use even in the premature infants.

Another object of the invention is to develop a skin-cleansing product, which is packaged as a disposable product and which maintains its sterility as long as its package remains unopened.

The structural and characteristic features and all the advantages of the invention will be more clearly understood from the detailed description provided below. Consequently, the evaluation should be made taking said detailed description into consideration.

### DETAILED DESCRIPTION OF THE INVENTION

The invention relates to a skin-cleansing product, which contains no antimicrobial agent and is suitable for use even in the premature infants, and to a method for obtaining said product.

In this detailed description, the preferred alternatives of the skin-cleansing product and the method for obtaining said product according to the invention are described only to enable a better understanding of the subject matter, without leading to any limiting effect.

The skin-cleansing product according to the invention comprises in its most basic form the water and at least one type of nonwoven fabric to which said water is applied. Said skin-cleansing product does not contain any antimicrobial agent intended to eliminate and/or prevent the growth of the microorganisms.

Said skin-cleansing product is preferably, but not limited to, a wet wipe. Compared with the alternatives of the state of the art, that it does not contain any antimicrobial agent is among the most important properties of this product. It is desired that the purity and sterility of the skin-cleansing product are preserved even though it does not contain any antimicrobial agent. For this reason, it is desired that the pH value of the product according to the invention is kept between 3 and 6 so that any microorganism and/or mold growth is prevented. In a preferred embodiment of the invention, the pH value is between 4 and 5. For this purpose, in the preferred embodiment of the invention, at least one type of pH-regulating agent is used in a quantity sufficient to bring the pH value into the desired range. In the product according to the invention, at least one type of pH-regulating agent selected from but not limited to the weak acids or strong acids or weak bases or strong bases or conjugated acid-base pairs is used as the pH-regulating agent.

Within the scope of the invention, it is possible to use at least one pH-regulating agent selected from at least one acid selected from citric acid, ascorbic acid and lactic acid as the weak acid and/or hydrochloric acid as the strong acid and/or at least one base selected from sodium citrate, sodium ascorbate and sodium lactate as the weak base and/or sodium hydroxide as the strong base and/or at least one conjugated acid-base pair selected from sodium citrate-citric acid or ascorbic acid-sodium ascorbate or sodium lactate-lactic acid.

The invention comprises the water in addition to the pH-regulating agent. Preferably, the pure water (the pure water with properties compliant with the pharmacopeia monographs) is used in order to obtain a sterile skin-cleansing product suitable for use in the infants. While the use of pure water may be preferred as the water, it is also possible to prefer the water purified via methods including but not limited to reverse osmosis, distillation and thermal or non-thermal filtration, or the drinking water.

The product developed within the scope of the invention preferably comprises the water at a ratio of 96-99% by weight.

A preferred embodiment of the invention comprises at least one pH-regulating agent at a ratio of 0.5-3% by weight.

A preferred embodiment of the invention preferably comprises 96-99% by weight water, 1-1.5% by weight at least one pH-regulating agent, and 0.5-1% by weight another pH-regulating agent different from the first one. In said embodiment, the pH-regulating agent used at a ratio of 1-1.5% by weight is preferably sodium citrate, the other pH-regulating agent used at a ratio of 0.5-1% by weight is preferably citric acid, and the water used is preferably the pure water. In said embodiment, the ratio between the weights of the pH-regulating agents used, i.e. the ratio of sodium citrate/citric acid, should be preferably 1:1.5.

Another alternative embodiment of the invention preferably comprises 96-99% by weight water, 1-1.5% by weight at least one pH-regulating agent, and 1-1.5% by weight another pH-regulating agent different from the first one. In said embodiment, the pH-regulating agents used are preferably sodium lactate and lactic acid, and the water used is preferably the pure water. In said embodiment, the ratio between the weights of the pH-regulating agents used, i.e. the ratio of sodium lactate/lactic acid, should be preferably 1:1.

This composition prepared for use in a skin-cleansing product, which is intended to be suitable for the skin of the infants and sterile, does not contain any additional chemical preservative. The composition comprising at least one pH-regulating agent and/or water is applied to at least one type of nonwoven fabric that the wet wipe according to the invention comprises as its raw material. Said nonwoven fabric is made of at least one material or a mixture of several materials selected from but not limited to preferably cotton, viscose, lyocell, paper pulp, polyester, natural and/or artificial silk, linen, wool, acetate, polypropylene, nylon 6.6, nomex, kevlar and elastane.

The skin-cleansing product developed within the scope of the invention is preferably packed in disposable packages. The package used features the sealing to enable the skin-cleansing product to remain sterile throughout its shelf life. A preferred number of skin-cleansing products may be placed in the package. Since the skin-cleansing product does not contain any antimicrobial agent, once the package has been opened, it is necessary to consume the entire product in said package and dispose of the product that has not been consumed without reusing the same.

Various available methods may be employed to obtain the skin-cleansing product in a sterile form. While the skin-cleansing product may be prepared with sterile materials in an aseptic environment, it is also possible to obtain the same by first preparing it in non-sterile form and sterilizing it before and/or during and/or after the packaging.

The method for obtaining the skin-cleansing product according to the invention in an aseptic environment comprises the process steps of
sterilizing the water and/or the nonwoven fabric,
sterilizing the packaging,
soaking the nonwoven fabric with water, and
packing the product with sterile packaging material.

The water to be used is sterilized preferably via the methods of thermal sterilization and/or sterile filtration, though said methods not being limited to these alternatives. In the embodiments where the sterile filtration is preferred, a membrane filter with the size of 0.22 μm is used. The saturated water vapor and/or dry air may be used for the sterilization with heat (thermal sterilization).

In cases where an aseptic environment is preferred, it is necessary to sterilize prior to manufacture all the equipment and packaging materials to be used, preferably by employing a method selected from but not limited to at least one of thermal sterilization or ionizing radiation methods, ethylene oxide gas, hydrogen peroxide gas and chlorine dioxide gas and/or at least one of other available sterilization methods. The sterilization of the nonwoven fabric to be used is achieved preferably via at least one method selected from but not limited to the methods of sterilization by the ultraviolet rays, ionizing radiation, ethylene oxide gas and/or hydrogen peroxide gas. Afterwards, the sterile nonwoven fabric is soaked with water, which is also sterile, and is packed by means of the sterilized equipment.

In case it is preferred to prepare the product in a non-sterile environment, all the production stages are performed without the necessity of sterility and the product according to the invention is subsequently sterilized before and/or during and/or after the packaging preferably via a means selected from but not limited to the heat (thermal) sterilization, for which the saturated water vapor and/or dry air may be used, and/or the ionizing radiation.

In the embodiments where the saturated water vapor is used for the heat (thermal) sterilization process, preferably the vapor temperature is kept in the range of 110-130° C. and/or the pressure is kept in the range of 1.9-2.2 bars. The sterilization process is performed such that it takes at least one hour.

In the embodiments where the ionizing radiation is employed for the sterilization process, the gamma rays are preferably used. The ray intensity for achieving the sterilization is in the value range of 3-15 kilograys (kGy). In case this value range is exceeded, the likelihood for the color and/or texture of the nonwoven fabric to be damaged comes into question. In a preferred embodiment of the invention, the ray intensity is in the range of 4-10 kGy.

It is possible to change the order of the process steps included in the method for obtaining the skin-cleansing product according to the invention or carry out these process steps simultaneously, based on the preferred embodiment.

Owing to the disposable sterile skin-cleansing product developed within the scope of the invention, which is free of antimicrobial preservative, and to the method for the production of this product, said problems and drawbacks of the state of the art encountered by the users were enabled to be eliminated.

1. A sterile skin-cleansing product suitable for use even in the neonates and/or premature infants wherein:
the skin-cleansing product does not contain any antimicrobial agent for killing microorganisms and/or preventing the growth of microorganisms;
the skin-cleansing product comprises water at a ratio of 96-99% by weight and at least one type of nonwoven fabric to which the water is applied; and
the skin-cleansing product comprises at least one pH-regulating agent at a ratio of 0.5-3% by weight.

2. (canceled)

3. A skin-cleansing product according to claim 1, wherein the skin-cleansing product comprises at least one type of weak acid selected from citric acid, ascorbic acid and lactic acid and/or at least one type of strong acid selected from hydrochloric acid and/or at least one type of weak base selected from sodium citrate, sodium ascorbate and sodium lactate and/or at least one type of strong base selected from sodium hydroxide as the pH-regulating agent.

4. (canceled)

5. A skin-cleansing product according to claim 1, wherein the skin-cleansing product comprises at least one conjugated acid-base pair selected from sodium citrate-citric acid or ascorbic acid-sodium ascorbate or sodium lactate-lactic acid as the pH-regulating agent.

6. (canceled)

7. A skin-cleansing product according to claim 5, wherein the weight ratio between sodium citrate and citric acid is 1:1.5.

8. A skin-cleansing product according to claim 5, wherein the weight ratio between sodium lactate and lactic acid is 1:1.

9. A skin-cleansing product according to claim 1, wherein the skin cleansing product comprises the pH-regulating agent in a quantity sufficient to bring the pH value preferably to the level of 4-5.

10. (canceled)

11. (canceled)

12. (canceled)

13. A skin-cleansing product according to claim 1, wherein the skin-cleansing product comprises at least one of or a mixture of several of cotton, viscose, lyocell, paper pulp, polyester, natural and/or artificial silk, linen, wool, acetate, polypropylene, nylon 6.6, nomex, kevlar and elastane fabric as the nonwoven fabric.

14. (canceled)

15. (canceled)

16. (canceled)

17. (canceled)

18. A skin-cleansing product according to claim 1, wherein the skin-cleansing product comprises:
pure water at a ratio of 96-99% by weight as the water,
sodium citrate a ratio of 1-1.5% by weight as the pH-regulating agent, and
citric acid at a ratio of 0.5-1% by weight as another pH-regulating agent.

19. (canceled)

20. A skin-cleansing product according to claim 1, wherein the skin-cleansing product comprises:
pure water at a ratio of 96-99% by weight as the water,
sodium lactate at a ratio of 1-1.5% by weight as the pH-regulating agent, and
lactic acid at a ratio of 1-1.5% by weight as another pH-regulating agent.

21. (canceled)

22. A method for obtaining a sterile skin-cleansing product suitable for use even in the neonates and/or premature infants wherein the skin-cleansing product is prepared with sterile materials in an aseptic environment or the skin-cleansing product is prepared in a non-sterile manner and is sterilized before and/or during and/or after the packaging.

23. A method according to claim 22 characterized in that the method for obtaining the product in an aseptic environment comprises the process steps of:
sterilizing the water via the methods of heat (thermal) sterilization and/or sterile filtration and/or sterilizing the nonwoven fabric via at least one method selected from sterilization by the ultraviolet rays or ionizing radiation or ethylene oxide gas and/or hydrogen peroxide gas,
sterilizing the packaging by the use of at least one method selected from heat (thermal) sterilization or ionizing radiation methods or ethylene oxide gas or hydrogen peroxide gas or chlorine dioxide gas,
soaking the nonwoven fabric with water, and
packing the product with sterile packaging material.

24. (canceled)

25. (canceled)

26. (canceled)

27. (canceled)

28. (canceled)

29. (canceled)

30. A method for obtaining a skin-cleansing product according to claim 23, wherein the saturated water vapor at a temperature of 110-130° C. and/or at a pressure of 1.9-2.2 bars and/or dry air is used in the heat (thermal) sterilization method.

31. (canceled)

32. A method for obtaining a skin-cleansing product according to claim 23, wherein gamma rays the ray intensity of which is in the range of 4-10 kGy, are used in the ionizing radiation method.

33. (canceled)

34. A method for obtaining a skin-cleansing product according to claim 22 wherein the product prepared in a non-sterile manner is sterilized before and/or during and/or after the packaging via a means selected from the heat sterilization, in which saturated water vapor and/or dry air is/are employed, and/or the ionizing radiation.

35. A skin-cleansing product according to claim 1, wherein the skin-cleansing product is a sterile wet wipe.

36. (canceled)
